(19) United States
(12) Patent Application Publication
(10) Pub. No.: US 2024/0058064 A1
(43) Pub. Date: Feb. 22, 2024

(54) COMPUTER IMPLEMENTED METHOD FOR AUGMENTED REALITY SPINAL ROD PLANNING AND BENDING FOR NAVIGATED SPINE SURGERY

(71) Applicant: Brainlab AG, Munich (DE)

(72) Inventors: Manfred Weiser, Munich (DE); Jörg Uhde, Munich (DE)

(21) Appl. No.: 18/271,305

(22) PCT Filed: Jan. 29, 2021
(86) PCT No.: PCT/EP2021/052181

§ 371 Date: Jul. 7, 2023

(51) Int. Cl. A61B 34/10; A61B 34/20; A61B 17/70; A61B 90/00

(52) U.S. Cl. CPC A61B 34/10; A61B 34/20; A61B 17/7013; A61B 90/36; A61B 2017/568

(57) ABSTRACT

Disclosed is a computer-implemented method for augmented reality spinal rod planning and bending for navigated spine surgery. A proposed spinal rod is determined that is a virtual model of a spinal rod with a desired shape. The proposed spinal rod is determined based on acquired positions of a plurality of spinal screws disposed on a spine of a patient. The spinal screws are configured for receiving a spiral rod interconnecting the plurality of spinal screws. Furthermore, the spinal rod itself is calibrated for tracking by a medical navigation device. This allows displaying the proposed spinal rod by an augmented reality device, thereby overlaying the tracked spinal rod with the proposed spinal rod. Thus, the proposed method inter alia provides the surgeon with improved information about the bending state of the spinal rod.

### FIELD OF THE INVENTION

The present invention relates to a computer-implemented method for augmented reality spinal rod planning and bending for navigated spine surgery, a medical navigation device and a corresponding computer program.

### TECHNICAL BACKGROUND

In spine surgery, spinal rods are used as implants for stabilization surgery of the human spine. Upon insertion of spinal screws into the spine of the patient, the spinal screws are interconnected by the spinal rods, spanning over the length of the vertebrae to be stabilized on the respective side of the spine. The rods must be formed/bent such that they fit through the heads of the spinal screws.

Currently, spinal rod bending is done intraoperatively, based on rough estimates, in particular by the surgeon's eye, and trial and error, and thus often a time consuming trial-and-error procedure.

Alternatively, a rod bending device relies on the input of measured screw head positions and proposes a rod bending which can be accomplished with a physical device.

Consequently, there is a need for more guidance of the surgeon when planning and bending the spinal rod.

The present invention has the object of providing an improved method for augmented reality spinal rod planning and bending for navigated spine surgery.

The present invention can be used for spinal stabilization procedures e.g. in connection with a system for image-guided surgery such as the Spine & Trauma Navigation System, a product of Brainlab AG.

Aspects of the present invention, examples and exemplary steps and their embodiments are disclosed in the following. Different exemplary features of the invention can be combined in accordance with the invention wherever technically expedient and feasible.

### Exemplary Short Description of the Invention

In the following, a short description of the specific features of the present invention is given which shall not be understood to limit the invention only to the features or a combination of the features described in this section.

A computer-implemented method for augmented reality spinal rod planning and bending for navigated spine surgery is presented.

In particular, in this method, a proposed spinal rod is determined that is a virtual model of a spinal rod with a desired shape. The proposed spinal rod is determined based on acquired positions of a plurality of spinal screws disposed on a spine of a patient. The spinal screws are configured for receiving a spiral rod interconnecting the plurality of spinal screws. Furthermore, the spinal rod itself is calibrated for tracking by a medical navigation device. This allows displaying the proposed spinal rod by an augmented reality device, thereby overlaying the tracked spinal rod with the proposed spinal rod. Thus, the proposed method inter alia provides the surgeon with improved information about the bending state of the spinal rod.

### General Description of the Invention

In this section, a description of the general features of the present invention is given for example by referring to possible embodiments of the invention.

This is achieved by the subject-matter of the independent claims, wherein further embodiments are incorporated in the dependent claims and the following description.

The described embodiments similarly pertain to the method for augmented reality spinal rod planning and bending for navigated spine surgery, the system for spinal rod planning and bending and a corresponding computer program. Synergetic effects may arise from different combinations of the embodiments although they might not be described in detail hereinafter. Furthermore, it shall be noted that all embodiments of the present invention concerning a method might be carried out with the order of the steps as explicitly described herein. Nevertheless, this has not to be the only and essential order of the steps of the method. The herein presented methods can be carried out with another order of the disclosed steps without departing from the respective method embodiment, unless explicitly mentioned to the contrary hereinafter.

Technical terms are used by their common sense. If a specific meaning is conveyed to certain terms, definitions of terms will be given in the following in the context of which the terms are used.

According to an aspect of the present disclosure, a computer implemented method for augmented reality spinal rod planning and bending for navigated spine surgery, comprises the following steps: In a step, a position of a plurality of spinal screws disposed on a spine is acquired, wherein the plurality of spinal screws are configured for receiving a spinal rod interconnecting the plurality of spinal screws. In another step, a proposed spinal rod is determined, being a virtual model of a spinal rod with a desired shape, using the acquired position of the plurality of spinal screws. In another step, the spinal rod is calibrated for tracking the spinal rod by a medical navigation device. In another step, the proposed spinal rod is displayed, by an augmented reality device, thereby overlaying the tracked spinal rod with the proposed spinal rod.

The term “spinal rod”, as used herein, relates to an implant used for stabilization of the human spine. The spinal rod in general is an elongated cylindrical rod that is bent into a shape that allows a surgeon to attach the spinal rod to the spine of a patient with the help of spinal screws.

The term “proposed spinal rod”, as used herein, comprises a virtual model of a spinal rod that reflects a spinal rod that has already been bent ideally for the spine surgery. In other words, the proposed spinal rod is a digital template for a spinal rod in accordance with the spine surgery. The proposed spinal rod is determined based on a predetermined model of a spinal rod, for example a standardized unbent spinal rod. Alternatively, the proposed spinal rod is determined based on a spinal rod model, being a virtual model of the spinal rod that needs to be planned and bent.

The term “spinal screw”, as used herein, comprises any kind of spinal bone screws, like pedicle screws, lateral mass screws or SAI screws. The spinal screws are directly connected with the spine of the patient, for example by being drilled into the spine.

Preferably, the augmented reality device comprises augmented reality glasses that in particular comprise at least one 3D scanner.

Preferably, calibrating the spinal rod, in particular by a calibration device of a medical navigation device, comprises determining the position and/or the shape of the spinal rod in the space. Thus, the position of the spinal rod and the proposed spinal rod, in particular the shape of the proposed spinal rod, is known. Further preferably, displaying the proposed spinal rod, by an augmented reality device, comprises overlaying the tracked spinal rod with the proposed spinal rod using the determined position and/or shape of the spinal rod in the space. Consequently, in order to support a surgeon in bending the spinal rod, the proposed spinal rod is displayed to the surgeon by the augmented reality device. As, due to the tracking of the spinal rod, the position and in particular the shape of the spinal rod in the space is known, the augmented reality device is configured to overlay the tracked spinal rod with the proposed spinal rod. In other words, the proposed spinal rod is displayed in the field of view of the surgeon in such a way that the surgeon always sees the proposed spinal rod in a specific spatial relationship to the spinal rod. For example, a left end of the spinal rod is always overlaid with a left end of the proposed spinal rod. The type of overlaying the spinal rod with the proposed spinal rod is preferably dynamically adjustable. Preferably, overlaying the spinal rod with the proposed spinal rod comprises displaying the proposed spinal rod in a spatial relationship to the spinal rod using the determined position and/or shape of the spinal rod.

Preferably, the method comprises the step of tracking the spinal rod, in particular by the medical navigation device, further in particular by a tracking device of the medical navigation device.

The augmented reality device is preferably comprised by the medical navigation device.

Preferably, calibrating the spinal rod comprises determining a spinal rod model, being a virtual representation of the spinal rod. In other words, during calibration of the spinal rod, the actual shape of the spinal rod is determined. Thus, any kind of spinal rod, un-bent or already pre-bent, is usable for the augmented reality device to display the proposed spinal rod over the real spinal rod in the field of view of the surgeon.

Preferably, the proposed spinal rod is determined using the spinal rod model and the position of the plurality of spine screws. In other words, the spinal rod model is digitally adjusted, or in other words a bending is simulated, using the position of the plurality of spine screws to determine the proposed spinal rod. Thus, the proposed spinal rod reflects the calibrated spinal rod in a bent shape that is ideal for the spine surgery.

Determining the proposed spinal rod using the acquired position of the plurality of spinal screws allows minimizing the user interaction, in particular the work of the surgeon.

Overlaying the tracked spinal rod with the proposed spinal rod with an augmented reality device allows the surgeon to bend the spinal rod in front of his eyes or in other words without controlling his progress of bending on a separate control screen showing the proposed spinal rod. Thus, the surgeon sees the spinal rod that has to be bent through the augmented reality device and also sees the virtual proposed spinal rod that is displayed in the field of view of the surgeon by the augmented reality device.

Thus, an improved method for augmented reality spinal rod planning and bending for navigated spine surgery is provided.

In the following preferred embodiments will be described in more detail.

In a preferred embodiment, the proposed spinal rod comprises a shape matching the position of the plurality of spinal screws on the spine.

The term “shape”, as used herein, also refers to the dimension and length of the spinal rod.

In a case, in which the shape of the spine of the patient should be reinforced by the spinal rod, the spinal rod has to be formed into a shape that matches the actual spine of the patient. The position of the spinal screws, or in other words the arrangement of the plurality of spinal screws on the spine, defines the shape of the proposed spinal rod. The same applies to the length of the proposed spinal rod. Thus, the proposed spinal rod represents a spinal rod that is ideally shaped and has the ideal length for its purpose of reinforcing the spine, in particular the shape of the spine.

Preferably, the shape of the spinal rod is digitized, for example by a 3D-scanner, to determine a spinal rod model. This spinal rod model is then preferably used to determine the proposed spinal rod. This allows using any kind of spinal rod of any shape or dimension, in particular a pre-bent spinal rod.

Thus, an improved method for augmented reality spinal rod planning and bending for navigated spine surgery is provided.

In a preferred embodiment, the method comprises the step of determining the proposed spinal rod using the acquired position of the plurality of spinal screws and a planned shape of the spine.

In a case, in which the shape of the spine of the patient should not only be reinforced but also adjusted or in other words corrected, the spinal rod has to be formed into a shape that matches the spine of the patient as it should be achieved by the surgery.

The planned shape of the spine in other words is the shape of the spine that should be achieved by the surgery.

In other words, the actual position of the spinal screws is combined with additionally planned corrections that should be applied to the spine. Combining the position of the plurality of spinal screws with the planned shape of the spine preferably comprises an, overlaying or adding the position of the plurality of spinal screws and the planned shape of the spine.

Preferably, the planned shape of the spine is determined by using a predetermined surgical plan. In particular, a planning software uses the position of the plurality of spinal screws and the planned shape of the spine to automatically determine the proposed spinal rod.

In other words, the position of the plurality of spinal screws is analysed in view of the planned shape of the spine. The position of the plurality of spinal screws is in particular provided to the planning software by digitalizing the plurality of spinal screws. The planned shape of the spine is in particular provided to the planning software by extraction from the predetermined surgical plan. Based on this analysis, which in particular comprises a comparison between the position of the plurality of spinal screws and the planned shape of the spine, the proposed spinal rod is automatically determined.

Further preferably, a surgeon manually determines the proposed spinal rod by using the planned shape of the spine and the position of the plurality of spinal screws.

In other words, the planning software is provided with the position of the plurality of spinal screws. The position of the plurality of spinal screws is visualized for the surgeon by the planning software. In particular, the planning software provides a provisional proposed spinal rod based on the position of the plurality of spinal screws. The surgeon analyses the position of the plurality of spinal screws in view of the planned shape of the spine. By using the planning software, the surgeon manually determines the proposed spinal rod, in particular by adjusting the provided provisional proposed spinal rod. For example, the surgeon adds further lordosis to the provisional proposed spinal rod or the position of the plurality of spinal screws.

In other words, the shape of the spinal rod can be corrected even more than the curvature represented by the digitized position of the spinal screws or the proposed spinal rod generated by the planning software.

For example, the surgeon adds a few more degree “lordosis”, for example in the planning software by manipulating the proposed spinal rod or displayed spine. Alternatively, a software interface saying “add/remove further lordosis to digitized screws” can be selected.

Afterwards the surgeon is preferably guided to bend the spinal rod into this new “virtual” position that is represented by the proposed spinal rod.

Consequently, in surgery the desired shape, for example with the additional lordosis, is then finally introduced to the spine by the bent spinal rod. In other words, the spinal rod pulls the anatomy, in particular the vertebrae of the spine of the patient, in the desired position.

Thus, an improved method for augmented reality spinal rod planning and bending for navigated spine surgery is provided.

In a preferred embodiment, calibrating the spinal rod comprises determining a spinal rod model, being a virtual representation of the spinal rod. The method comprises the step of determining support data using the spinal rod model; wherein the support data comprises information linked to the spine. The method further comprises the step of overlaying, by the augmented reality device, the spinal rod with the support data.

The term “support data”, as used herein, represents information of the spine as it applies to the present spine. However the support data preferably also represents information of the spine as it applies to the spine when the spinal rod is connected with the plurality of spinal screws. In other words, the support data contains information or in other words parameters of the spine for the surgeon or the planning software relating to the present shape of the spine or relating to the planned shape of the spine.

Preferably, the support data is used by the planning software or the surgeon via the planning software to adjust the proposed spinal rod. In other words, the support data provides thresholds for different parameters relating to the spine of the patient that have to be considered when adjusting the shape of the proposed spinal rod.

Thus, the surgeon is provided by the augmented reality device with additional information concerning the planning and bending of the spinal rod.

Thus, an improved method for augmented reality spinal rod planning and bending for navigated spine surgery is provided.

In a preferred embodiment, determining the spinal rod model, comprises recognizing the shape of the spinal rod in relation to a tracked reference array.

In other words, the spinal rod is calibrated by detecting the shape of the spinal rod, in particular by a 3D camera or a 3D laser scanner device, and detecting the tracked reference array. The detected shape of the spinal rod is used to determine the spinal rod model, representing the spinal rod. Due to the also detected reference array, in particular comprising reference markers, a position of the spinal rod in the space is known.

Preferably, the augmented reality device is configured for acquiring a surface model of the spinal rod based on which the spinal rod model is determined. In particular, correlated video images of optical channels of the augmented reality device allow for a surface reconstruction of the spinal rod. Thus, the augmented reality device is configured for determining the spinal rod model.

Preferably, the spinal rod is calibrated using a calibration block or pre-calibration data, if the length of the spinal rod is known, that is adjusted by the detected position of the tracked reference array.

Thus, an improved method for augmented reality spinal rod planning and bending for navigated spine surgery is provided.

In a preferred embodiment, determining the spinal rod model, comprises acquiring the shape of the spinal rod by a tracking device.

Preferably, the tracking device comprises a tracked pointer, wherein the position in the space of the tracked pointer is known. The spinal rod, in particular a plurality of surface points of the spinal rod, are sampled by the tracking device to calibrate the spinal rod and determine the spinal rod model. Preferably, the tracking device is slid along at least part of the surface of the spinal rod to sample the spinal rod. Preferably, the tracking device comprises a tracking pointer with a specific shaped tip, for example a ring shaped tip or a half-pipe-shaped tip.

Thus, an improved method for augmented reality spinal rod planning and bending for navigated spine surgery is provided.

In a preferred embodiment, the method comprises the step of dynamically adjusting the spinal rod model using the tracked spinal rod.

In other words, the shape of the spinal rod is continuously detected, for example by a 3D camera and the spinal rod model is adjusted using the continuously detected shape of the spinal rod. Thus, the spinal rod model is always up to date compared to the spinal rod. Consequently, during bending the spinal rod, the change in shape of the spinal rod due to bending is directly reflected by the spinal rod model. In other words, the shape of the spinal rod model is congruent to the shape of the spinal rod.

Preferably, the spinal rod model is adjusted in real-time.

Consequently, the support data that is determined using the spinal rod model also reflects the changes in shape of the spinal rod. For example, when the support data comprises different bending indicators, indicating the spot on which the spinal rod should be bent, any bending indicator that has already been acknowledged by bending the spinal rod is discarded and not displayed anymore.

Thus, an improved method for augmented reality spinal rod planning and bending for navigated spine surgery is provided.

In a preferred embodiment, the support data comprises at least one bending indicator, determined by using the proposed spinal rod and the spinal rod model.

Preferably, the bending indicator indicates a spot on the spinal rod on which the spinal rod should be bent by the surgeon. Thus, the surgeon is guided to bend the spinal rod to arrive at the proposed spinal rod in an improved way. The bending indicators are preferably displayed directly overlapping on the spinal rod. For example, the bending indicator comprises a marker like a dot or a vertical line.

Preferably, the at least one bending indicator comprises an order of bending. For example, the at least one bending indicator is numbered to indicate the surgeon, in which order the spinal rod should be bent to ideally arrive at the proposed spinal rod.

Thus, the surgeon is provided with improved guidance within the point of view of the surgeon while bending the spinal rod.

Thus, an improved method for augmented reality spinal rod planning and bending for navigated spine surgery is provided.

In a preferred embodiment, the method comprises the following steps. Determining a spine model, being a virtual representation of the spine and adjusting the spine model using the spinal rod model. The support data comprises a spine indicator, determined by using the spine model, indicating the spine on the spinal rod.

Preferably, the spine model is determined using patient specific spine data that is in particular predetermined. For example, the patient specific spine data is determined by using image segmentation techniques, like for example atlas and/or artificial intelligence methods, for detecting the vertebrae in available image data of the patient's spine. The image data may be preoperative or intraoperative image data. The image data preferably comprises 3D datasets, like CT datasets or MRT datasets. Alternatively, the image data comprises 2D or 3D X-ray images, allowing for an approximate reconstruction of the spinal shape. The image data is preferably model-enhanced, in particular comprising morphing of models into detected outlines in the X-ray images.

The image data for example only comprises one X-ray image together with segmentation techniques, as long as an adjustment of the spine model would be visible from the angle the X-ray indicates the spine.

The spine indicator thus indicates the surgeon in this field of view how an adjustment of the spinal rod impacts a deformation of the spine.

Additionally, the spine model is displayed in a different angle than the proposed spinal rod model to the surgeon. In many cases, the spine model is only displayed to the surgeon from the top of the spine making an adjustment of the spine model hardly visible to the surgeon. So, the spine model is also displayed form a side angle of the spine, in particular not overlapping the spinal rod, but still in the field of view of the surgeon. For example, the spine model is displayed in a corner of the field of view of the surgeon.

Thus, an improved method for augmented reality spinal rod planning and bending for navigated spine surgery is provided.

In a preferred embodiment, the method comprises the step of determining a spinal screw model, being a virtual representation of the plurality of spinal screws disposed on the spine. The support data comprises at least one screw indicator, determined by using the position of the plurality of spinal screws, indicating the plurality of spinal screws on the spinal rod.

In other words, the at least one screw indicator represents a digitized model of a spinal screw, in particular at the determined position of the spinal screw.

Preferably, the at least one screw indicator is displayed on the proposed spinal rod.

When the at least one screw indicator is displayed on the proposed spinal rod, the at least one screw indicator either represents the plurality of spinal screws disposed on the spine as they are positioned in reality, or represents the plurality of spinal screws disposed on the spine as they are planned to be positioned due to an adjustment of the spine.

In other words, initially the at least one screw indicator is determined by using the position of the plurality of spinal screws and thus reflects the reality of shape and position of the spinal screw on the spine. If the surgeon however adjusts the proposed spinal rod, in particular by determining a planned shape of the spine, the position of the at least one spinal screw indicator is also adjusted accordingly.

Thus, the at least one screw indicator dynamically indicates the shape and position of the plurality of spinal screws in line with the proposed spinal rod. Preferably, the surgeon is constantly provided with information about the shape and position of the plurality of spinal screws as they would be arranged on the planned shape of the spine.

Thus, the surgeon is provided with constant feedback how the planned adjustment of the spine impacts the arrangement of the spinal screws in particular indicated on the spine indicator. Furthermore, the spinal screw model is preferably used by the planning software when determining the proposed spinal rod.

In addition, when planning the spinal rod, in particular when determining the proposed spinal rod, the surgeon or the planning software virtually adjust a position of the spinal screws in regards to the spine, in particular independent from a certain planned alignment. This allows for example to increase the biomechanical strength or minimizes a skin cut size.

During spinal rod planning and bending, this allows the visualization of the impact of current bending to relative spinal screw positions and patient anatomy, enhancing the spinal rod planning and bending process.

Thus, an improved method for augmented reality spinal rod planning and bending for navigated spine surgery is provided.

In a preferred embodiment, the method comprises the step of determining forces applied to the plurality of spinal screws by using the spine model and the spinal rod model. The support data comprises a force indicator, determined by using the determined forces, indicating forces applied to the plurality of spinal screws, if the spinal rod would be connected to the spinal screws.

Preferably, the force indicator comprises a vector indicating the amount of applied force to the spine and/or the spinal screws.

Preferably, the forces are determined using finite elements methods, FEM methods, based on a bio-mechanical model, taking into account material properties of the spinal rod and the spinal screws.

The surgeon is thus provided with constant feedback how the planned adjustment of the spine impacts the forces applied to the plurality of spinal screws, if the spinal rod would be connected to the spinal screws. In other words, a specific planned adjustment of the spine might appear ideal, however might introduce a relative large amount of tension or stress to the spine or one or more spinal screws. With the force indicator, the surgeon is guided not to choose an adjustment of the spine that would introduce an unreasonable amount of force on the spine or one or more spinal screws when connecting the spinal rod to the spinal screws. Furthermore, the determined forces are used by the planning software to determine the proposed final rod, in particular by comparing the determined forces with predetermined thresholds.

This allows to prevent the loosening of the spinal screws in the spine due to the induced forces of the spinal rod.

Also, this ensures a mechanical stability of the rod construct, or in other words, the spinal rod connected to the spine with the spinal screws.

Thus, an improved method for augmented reality spinal rod planning and bending for navigated spine surgery is provided.

In a preferred embodiment, the method comprises the step of determining a force warning, if the determined forces exceed a predetermined threshold. The support data comprises a force warning indicator, determined by using the determined force warning.

Preferably, the force warning indicator comprises a colour code. In order to further guide the surgeon in the process of planning and bending the spinal rod, the determined forces are automatically compared with predetermined thresholds and the force warning indicator is displayed to the surgeon in this field of view to alarm the surgeon of an exceeding amount of force that would be applied to the spine or one or more spinal screws when connecting the spinal rod in line with the proposed spinal rod with the plurality of spinal screws.

In other words, the surgeon is actively alarmed, if the planned adjustment of the spine, introduced by the planned bending of the spinal rod, would lead to an unwanted amount of tension of the spine or between the spine and the plurality of spinal screws.

Thus, an improved method for augmented reality spinal rod planning and bending for navigated spine surgery is provided.

In a preferred embodiment, the method comprises the step of determining at least one anatomical parameter of the spine by using the spine model and the spinal rod model. The support data comprises at least one anatomical parameter indicator, determined by using the at least one determined anatomical parameter.

Preferably, the anatomical parameters comprise an inter-vertebral angle, in particular a cobb angle, a lordosis, a kyphosis, a scoliosis for sagittal and coronal balance, as well as an inter-vertebral distance or a distance of spondylolistheses. Preferably, the availability of the anatomical parameters depends on available information like number and location of imaged vertebrae.

The surgeon is thus provided with additional information that is directly displayed in the field of view of the surgeon. In case of an adjustment of the spine, the at least one parameter of the spine is also displayed for the proposed spinal rod.

This allows a prediction of outcome of anatomical parameters during planning of the spinal rod, or in other words during determination of the proposed spinal rod.

Thus, an improved method for augmented reality spinal rod planning and bending for navigated spine surgery is provided.

In a preferred embodiment, the method comprises the step of determining an average deviation between the spinal rod and the proposed spinal rod by using the spinal rod model and the proposed spinal rod. The support data comprises a deviation indicator, determined by using the determined average deviation.

In other words, the deviation indicator allows the surgeon to assess, how accurate the bending of the spinal rod has been performed and if the surgeon has to continue bending or is finished.

Thus, the surgeon is further guided in his pursue to bend the spinal rod.

Thus, an improved method for augmented reality spinal rod planning and bending for navigated spine surgery is provided.

In a preferred embodiment, calibrating the spinal rod comprises providing the spinal rod with a reference device, defining an origin of a spinal rod coordinate system and determining a spinal-rod-to-cam-coordinate-transformation, which describes a transformation between the spinal rod coordinate system and a camera coordinate system.

Preferably, the reference device is a reference star.

In other words, the origin of the spinal rod coordinate system is defined by the position of the reference device on the spinal rod.

In a preferred embodiment, acquiring the position of the plurality of spinal screws comprises recognizing the plurality of spinal screws by the augmented reality device.

Preferably, the position of the plurality of spinal screws is scanned by a 3D scanner integrated into the augmented reality device or image processing from a video recorded by the augmented reality device.

Further preferably, correlated images of the at least one video camera or 3D depth camera of the augmented reality device are used for the surface reconstruction of the spinal screws, in particular the screw heads, which are matched to a generic model or to manufacturer specific models from a database.

For example, the augmented reality device comprises a single RGB stereo camera and a time-of-flight camera that are used to acquire the position of the plurality of spinal screws.

Thus, the position of the plurality of spinal screws is acquired by the augmented reality device.

In a preferred embodiment, acquiring the position of the plurality of spinal screws comprises extracting of the position of the plurality of spinal screws from a planning application.

Preferably, the planning application comprises information about the shape of the spine and the spinal screws already disposed on the spine, in particular indicated by preoperative image data. In other words, spinal screws that are planned in preoperative image data are transferred after registration of these data into a patient coordinate system. The position and axial orientation of the spinal screws, in particular the spinal screw heads, are predetermined for monoaxial spinal screws. For polyaxial spinal screws, a best fit can be modelled.

Thus, the position of the plurality of spinal screws is acquired automatically from external.

In a preferred embodiment, acquiring the position of the plurality of spinal screws comprises detecting the position of the plurality of spinal screws in intraoperative image data.

Preferably, metal artefacts detected in paired registered 2D images or single registered 3D scans are matched to a generic model or to manufacturer specific models from a database for the spinal screws. As the image data is registered, the 3D position of the identified spinal screws is known in the patient coordinate system.

In a preferred embodiment, wherein acquiring the position of the plurality of spinal screws comprises calibrating each of the plurality of spinal screws by using a tracked pointer.

For example, a tip of the tracked pointer touches or pivots the centre of the spinal screw head to acquire the position of the spinal screw.

According to another aspect of the invention a medical navigation device is configured for executing the method, as described herein.

Preferably, the medical navigation device comprises an augmented reality device and a control unit. The augmented reality device is configured for acquiring a position of a plurality of spinal screws disposed on a spine, wherein the plurality of spinal screws are configured for receiving a spinal rod interconnecting the plurality of spinal screws. Further the augmented reality device is configured for calibrating the spinal rod for tracking the spinal rod by the medical navigation device. Further the augmented reality device is configured for displaying the proposed spinal rod, thereby overlaying the tracked spinal rod with the proposed spinal rod.

The control unit is configured for determining the proposed spinal rod, being a virtual model of a spinal rod, using the acquired position of the plurality of spinal screws.

According to another aspect of the invention a computer program which, when running on a computer or when loaded onto a computer, causes the computer to perform the method steps of the method, as described herein and/or a program storage medium on which the program is stored; and/or a computer comprising at least one processor and a memory and/or the program storage medium, wherein the program is running on the computer or loaded into the memory of the computer; and/or a data stream which is representative of the program.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable to planning and bending the spinal rod outside of the patient's body. For this reason alone, no surgical or therapeutic activity and in particular no surgical or therapeutic step is necessitated or implied by carrying out the invention.

Although the method might be executed intraoperatively, the steps of the method do not contain surgical or therapeutic activity.

### Definitions

In this section, definitions for specific terminology used in this disclosure are offered which also form part of the present disclosure.

Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

Registering

The n-dimensional image of a body is registered when the spatial location of each point of an actual object within a space, for example a body part in an operating theatre, is assigned an image data point of an image (CT, MR, etc.) stored in a navigation system.

Image Registration

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

Marker Holder

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

Pointer

A pointer is a rod which comprises one or more—advantageously, two—markers fastened to it and which can be used to measure off individual co-ordinates, for example spatial co-ordinates (i.e. three-dimensional co-ordinates), on a part of the body, wherein a user guides the pointer (for example, a part of the pointer which has a defined and advantageously fixed position with respect to the at least one marker attached to the pointer) to the position corresponding to the co-ordinates, such that the position of the pointer can be determined by using a surgical navigation system to detect the marker on the pointer. The relative location between the markers of the pointer and the part of the pointer used to measure off co-ordinates (for example, the tip of the pointer) is for example known. The surgical navigation system then enables the location (of the three-dimensional co-ordinates) to be assigned to a predetermined body structure, wherein the assignment can be made automatically or by user intervention.

Reference Star

A “reference star” refers to a device with a number of markers, advantageously three markers, attached to it, wherein the markers are (for example detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star for example features a way of being attached to the object (for example, a clamp and/or a thread) and/or a holding element which ensures a distance between the markers and the object (for example in order to assist the visibility of the markers to a marker detection device) and/or marker holders which are mechanically connected to the holding element and which the markers can be attached to.

Navigation System

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information.

Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

The invention also relates to a navigation system for computer-assisted surgery, comprising:
a computer for processing the absolute point data and the relative point data;
a detection device for detecting the position of the main and auxiliary points in order to generate the absolute point data and to supply the absolute point data to the computer;
a data interface for receiving the relative point data and for supplying the relative point data to the computer; and
a user interface for receiving data from the computer in order to provide information to the user, wherein the received data are generated by the computer on the basis of the results of the processing performed by the computer.

Surgical Navigation System

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

Shape Representatives

Shape representatives represent a characteristic aspect of the shape of an anatomical structure. Examples of shape representatives include straight lines, planes and geometric figures. Geometric figures can be one-dimensional such as for example axes or circular arcs, two-dimensional such as for example polygons and circles, or three-dimensional such as for example cuboids, cylinders and spheres. The relative position between the shape representatives can be described in reference systems, for example by co-ordinates or vectors, or can be described by geometric variables such as for example length, angle, area, volume and proportions. The characteristic aspects which are represented by the shape representatives are for example symmetry properties which are represented for example by a plane of symmetry. Another example of a characteristic aspect is the direction of extension of the anatomical structure, which is for example represented by a longitudinal axis. Another example of a characteristic aspect is the cross-sectional shape of an anatomical structure, which is for example represented by an ellipse. Another example of a characteristic aspect is the surface shape of a part of the anatomical structure, which is for example represented by a plane or a hemisphere. For example, the characteristic aspect constitutes an abstraction of the actual shape or an abstraction of a property of the actual shape (such as for example its symmetry properties or longitudinal extension). The shape representative for example represents this abstraction.

Referencing

Determining the position is referred to as referencing if it implies informing a navigation system of said position in a reference system of the navigation system.

Atlas/Atlas Segmentation

Preferably, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a patient's body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image—the atlas data therefore contains or is comparable to medical image data. Such a comparison can be carried out for example by applying an image fusion algorithm which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

Mapping

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### BRIEF DESCRIPTION OF THE DRAWINGS

In the following, the invention is described with reference to the appended figures which give background explanations and represent specific embodiments of the invention.

The scope of the invention is however not limited to the specific features disclosed in the context of the figures, wherein

FIG. 1 shows the medical navigation device used by a surgeon for planning and bending the spinal rod;

FIG. 2a shows a schematic view through the augmented reality device displaying the unbent spinal rod overlaid by the proposed spinal rod;

FIG. 2b shows a schematic view through the augmented reality device displaying the partially bent spinal rod overlaid by the proposed spinal rod;

FIG. 3 shows a schematic view of tracking the spinal rod by the medical navigation device;

FIG. 4 shows a schematic view of the medical navigation device;

FIG. 5a shows a schematic view through the augmented reality device displaying the unbent spinal rod overlaid by the proposed spinal rod and spinal screw indicators;

FIG. 5b shows a schematic view through the augmented reality device displaying the unbent spinal rod overlaid by the proposed spinal rod and bending indicators;

FIG. 6 shows a schematic view of a spine of a patient with spinal screws that are connected by a spinal rod; and

FIG. 7 shows a schematic view of the computer implemented method for augmented reality spinal rod planning and bending for navigated spine surgery.

### DESCRIPTION OF EMBODIMENTS

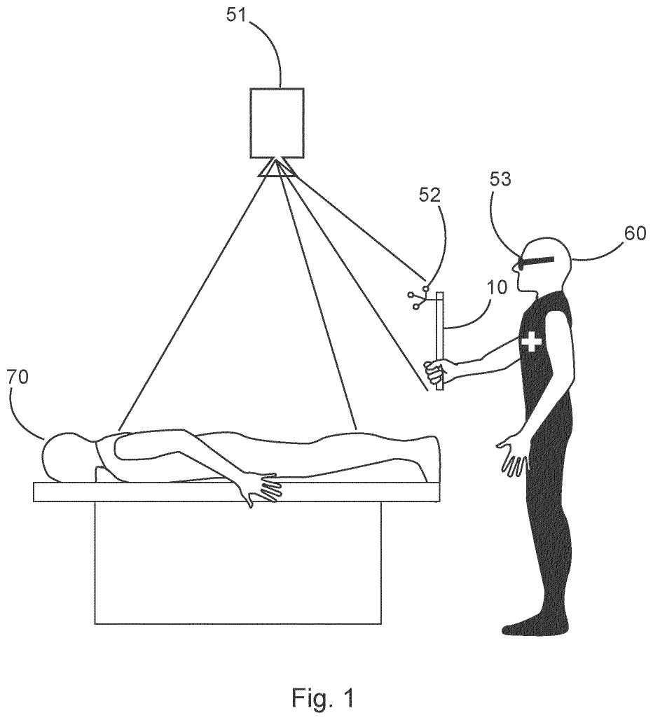

FIG. 1 shows the medical navigation device used by a surgeon 60 for planning and bending a spinal rod 10. The spinal rod 10 should be used in a spine surgery, in which the spine of a patient 70 is adjusted and/or reinforced by the spinal rod 10. For this purpose, the spine 40 is provided with a plurality of spinal screws. In the spine surgery, the spinal rod 10 is connected and attached to the spine 40 by the spinal screws 30. Thus, the spine 40 of the patient is reinforced or adjustments to the spine 40 are applied by the spinal rod 10.

However, before attaching the spinal rod 10 to the spine 40 of the patient, the spinal rod 10 has to be shaped accordingly, in particular by bending the spinal rod 10 into a desired shape that achieves the reinforcing and/or adjusting effects of the surgery. Although the bending itself is in general performed by a bending device, the bending device is usually manually operated by the surgeon 60.

On general, a proposed spinal rod 20, in other words a virtual model of a spinal rod 10 reflecting the desired shape of the spinal rod is displayed to the surgeon 60 at a separate screen. The surgeon then tries to bend the spinal rod 10 in the desired shape following the display of the proposed spinal rod 20.

In the illustrated case, the surgeon 60 uses a medical navigation device 50, that is usually also used in the spine surgery. The surgeon 60 wears an augmented reality device 53, in particular augmented reality glasses, that is part of the medical navigation device 50 and functions as a screen to display the proposed spinal rod 20.

The proposed spinal rod 20 itself is determined based on a position Ps of the plurality of screws 30. The position Ps of the plurality of screws 30 is for example acquired by a camera 51 of the medical navigation device 50. The camera 51 for example comprises a 3D camera configured for acquiring a shape and a position in space of the plurality of spinal screws 30.

Using the acquired position Ps of the plurality of screws 30, the medical navigation device 50 analyses an arrangement of the plurality of spinal screws 30 on the spine and determines the proposed spinal rod 20. The proposed spinal rod 20 in other words is a virtual model of the spinal rod 10 as it has to be shaped to fulfil its task in the spine surgery. In a first step, the shape of the proposed spinal rod 20 directly relates to the shape of the spine 40 of the patient 70. However, automatically by a planning software or manually by the surgeon 60, the shape of the proposed spinal rod 20 can be adjusted. In a case, in which the spine 40 of the patient 70 should not only be reinforced but adjusted, the shape of the proposed spinal rod 20 has to reflect such adjusted shape of the spine 40 of the patient as it should become through the spine surgery. For example, the surgeon adds a specific amount of Iordosis to the proposed spinal rod 20 via a user interface of the planning software to adjust the proposed spinal rod 20.

The planning software is preferably provided with support data Ds that are displayed to the surgeon 60. Thus, the surgeon virtually adjust the proposed spinal rod 20, in particular the spinal alignment of the patient 70, until a desired medical outcome is reached. The medical outcome is preferably indicated by the support data Ds, comprising surgery relevant parameters.

Consequently, the proposed spinal rod 20 is provided to the augmented reality device 53 to be displayed within the field of view of the surgeon. Thus, the surgeon always see the proposed spinal rod 20 in this field of view when holding the spinal rod 10 in his hands to bend the spinal rod 10 in accordance with the proposed spinal rod 20.

Furthermore, the spinal rod 10 itself is calibrated, for example by a calibration device of the medical navigation device 50. In this case, the spinal rod 10 comprises a reference device 52, attached to the spinal rod 10. Thus, performing a calibration, an acquired position of the spinal rod 10 is transformed into coordinates of the medical navigation device 50. In other words, the medical navigation device 50 learns about the position of the spinal rod 10 in its own coordinates. Thus, when displaying the proposed spinal rod 20 to the surgeon 60 via the augmented reality device 53, the augmented reality device 53 arranges the proposed spinal rod 20 in a way that overlaps the spinal rod 10 that the surgeon observes through the augmented reality device 53. By calibrating and continuously tracking the spinal rod 10, the augmented reality device 53 can always overlap the spinal rod 10 in the field of view of the surgeon 60 with the proposed spinal rod 20. This allows for an enhanced view of the proposed spinal rod 20 for the surgeon 60 when bending the spinal rod 10.

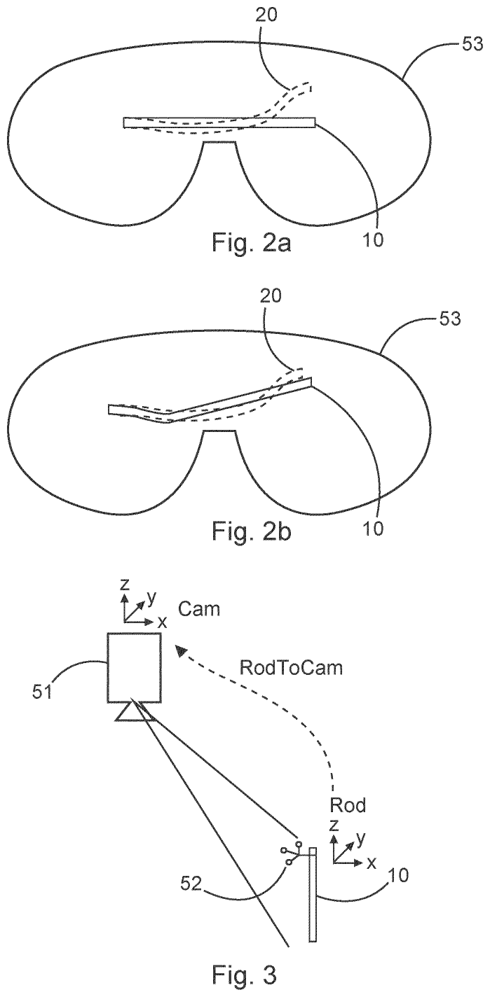

FIG. 2a shows a schematic view through the augmented reality device 53 displaying the unbent spinal rod 10 overlaid by the proposed spinal rod 20. In other words, the surgeon 60 has the spinal rod 10 in his field of view in order to bend the spinal rod 10 in a shape that is needed for the spinal surgery. The surgeon 60 wants to bend the spinal rod 10 in shape, in particular with the help of a bending tool, based on the proposed spinal rod 20. Thus, the proposed spinal rod 20 is displayed by the augmented reality device 53 in the field of view of the surgeon 60. The augmented reality device 53 not only randomly displays the proposed spinal rod 20 in the field of view of the surgeon, but displays the proposed spinal rod 20 in a way that overlaps the spinal rod 10 from the perspective of the surgeon 60. In this case, the augmented reality device 53 arranges the proposed spinal rod 20 such that the left end of the proposed spinal rod 20 matches the left end of the spinal rod 10. This allows for an improved display of information for the surgeon in order to bend the spinal rod 10.

FIG. 2b shows a schematic view through the augmented reality device 53 displaying the unbent spinal rod 10 overlaid by the proposed spinal rod 20. Compared to the spinal rod 10 in FIG. 2a, the spinal rod 10 has already been bent. The spinal rod 10 has either been pre-bent by the surgeon 60 from experience or has been pre-bent by the surgeon 60 with the help of the augmented reality device 53. As the spinal rod 10 is tracked by the medical navigation device 50, the spinal rod 10 does not have to be an unbent spinal rod 10 to be used by the medical navigation device 50. Any pre-bent spinal rod 10 can be calibrated and tracked by the medical navigation device 50 and be overlaid with the proposed spinal rod 20. In other words, the surgeon 60 has the partially bent spinal rod 10 in his field of view in order to finish bending the spinal rod 10 in the shape that is needed for the spinal surgery. Like in FIG. 2a, the proposed spinal rod 20 is displayed by the augmented reality device 53 in the field of view of the surgeon 60. The augmented reality device 53 not only randomly displays the proposed spinal rod 20 in the field of view of the surgeon, but displays the proposed spinal rod 20 in a way that overlaps the spinal rod 10 from the perspective of the surgeon 60. In this case, the augmented reality device 53 arranges the proposed spinal rod 20 such that the already bent part of the spinal rod 10 matches the corresponding part of the spinal rod 10. This allows the surgeon 60 to be sure that the already bent part of the spinal rod 10 satisfies the proposed spinal rod 20.

FIG. 3 shows a schematic view of tracking the spinal rod 10 by the medical navigation device 50. The spinal rod 10 is provided with a reference device 52, in this case a reference array of three markers. The reference device 52 marks the origin of a Rod-coordinate system Rod. The medical navigation device 50 comprises the camera 51, which marks the origin of a Cam-coordinate system Cam. The Cam-coordinate system Cam is known to the medical navigation device 50. When calibrating the spinal rod 10, for example by using a calibration device like a calibration block, a relationship between the Rod-coordinate system and the Cam-coordinate system is determined. This relationship is indicated by a spinal-rod-to-camera-coordinate-transformation RodToCam.

The term “transformation”, as used herein, specifically describes a translation and/or rotation between two objects like a tracking system of the medical navigation device 50 and a calibration device of the medical navigation device 50. As each object is represented by a location and orientation in space, preferably a coordinate system is defined for each object, so the transformation allows to describe coordinates of points in one system in terms of coordinates in another system. For example, a calibration point of the calibration device is given in local coordinates of the calibration device. Using a transformation from the calibration device to the spinal rod 10, the spinal rod 10 can be represented in calibration device coordinates. Every transformation has a unique reverse transformation, so the spinal rod coordinates can also be represented in calibration device coordinates. To optimize the meaning of coordinate systems, their origin is typically located at a point of interest within their object. A preferable implementation of such transformations is the usage of 4×4 matrices that are widely used in the field of computer graphics for exactly this purpose. Thus one transformation matrix can include translation and rotation, theoretically every affine transformation in 3D space, and it leaves the matrix invertible. A composition of transformations like calibration device to camera, then camera to spinal rod 10 is represented by a multiplication of the according matrices (in reverse order). A transformation between two coordinate systems can be set up by knowing the origin and three perpendicular axes of one coordinate system in the coordinates of the other coordinate system. For 4×4 matrices the commonly used technique is a change of basis where the axes are normalized and written into the upper left 3×3 part of the 4×4 matrix while the translation between the coordinate systems is taken into account in the 4^{th} column.

In a tracking setup for a calibration of the spinal rod 10, different coordinate systems of the participating objects of the tracking system need to be related to each other. In other words, the tracking system, in particular the camera 51, comprises a camera coordinate system Cam, the calibration device comprises a calibration device coordinate system, and the spinal rod 10 comprises a spinal rod coordinate system Rod at its marker array.

For calibrating the spinal rod 10, it is necessary to find a relationship between the spinal rod 10 and the calibration device. By holding the spinal rod 10 onto a known spot of the calibration device, this relationship can be determined. As it is assumed that the relationship between the camera coordinate system Cam and the calibration device coordinate system is known, the relationship between the camera coordinate system Cam and the spinal rod coordinate system Rod can be calculated.

The orientation of the instrument tip coordinate system is preferable pre-defined in relation to the instrument marker coordinate system. However, planes or other features of the calibration device can be used to specifically calibrate the axis of an instrument, which is not the main object of this invention.

Due to the spinal-rod-to-camera-coordinate transformation, the position and in particular the shape of the spinal rod 10 is always known to the medical navigation device 50.

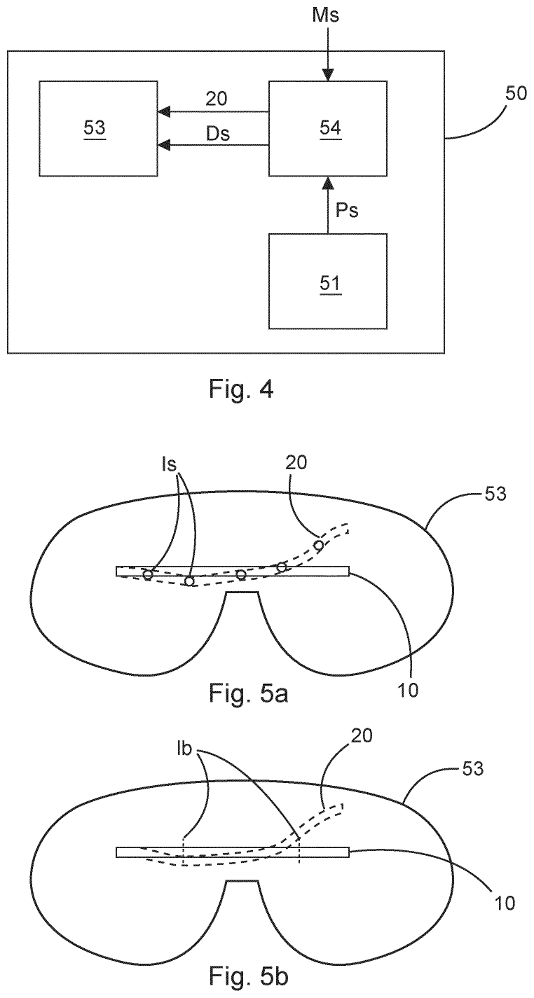

FIG. 4 shows a schematic view of the medical navigation device 50. The medical navigation device 50 comprises a camera 51 that is in particular configured for digitalizing the plurality of spinal screws 30 on the spine 40 of the patient 70, an augmented reality device 53 that functions as a display for the medical navigation device 50 and a control unit 54. The camera 51, in particular by using a tracked instrument, determines the position Ps of the plurality of spinal screws 30 and provides the position Ps to the control unit 54. The control unit 54 uses the position Ps of the plurality of spinal screws 30 to determine a proposed spinal rod 20, being a virtual model of the spinal rod 10 as it has to be shaped to fit the plurality of spinal screws 30. The proposed spinal rod 20 is provided to the augmented reality device 53, where the proposed spinal rod 20 is displayed to the surgeon as a template to bend the spinal rod 10 into shape. In addition to the proposed spinal rod 10, additional information can be provided by the control unit 54 to the augmented reality device 53. For example, the control unit 54 is provided with a spine model Ms, representing the spine 40 of the patient 70. The spine model Ms is for example used by the control unit 54 to determine support data Ds that is provided to the augmented reality device 53. The spine model Ms can be used to determine how the shape of the proposed spinal rod 20 affects forces on the spine 40 or the spinal screws 30. This information is then included into the support data Ds and used by the augmented reality device 53 to display the applied forces to the different objects. The surgeon wearing the augmented reality device 53 is thus provided with additional information on the case.

The control unit 54 preferably comprises a planning software, which allows the already determined proposed spinal rod 20 to be adjusted, automatically by the planning software itself, or manually by the surgeon 60 over an input interface.

FIG. 5a shows a schematic view through the augmented reality device displaying the unbent spinal rod 10 of FIG. 2a, overlaid by the proposed spinal rod 20 and spinal screw indicator Is. The spinal screw indicators Is are based on support data Ds that is in particular provided by the control device 54. The spinal screw indicators Is indicate where the plurality of spinal screws 30 are disposed on the spinal rod 10, when the spinal rod 10 has the shape of the proposed spinal rod 20.

FIG. 5b shows a schematic view through the augmented reality device displaying the unbent spinal rod 10 of FIG. 2a overlaid by the proposed spinal rod 20 and bending indicators Ib. The bending indicators Ib are based on support data Ds that is in particular provided by the control device 54. The bending indicators Ib are displayed overlaying the spinal rod 10, indicating the spots, where the spinal rod 10 has to be ideally bent to arrive at the shape of the proposed spinal rod 20.

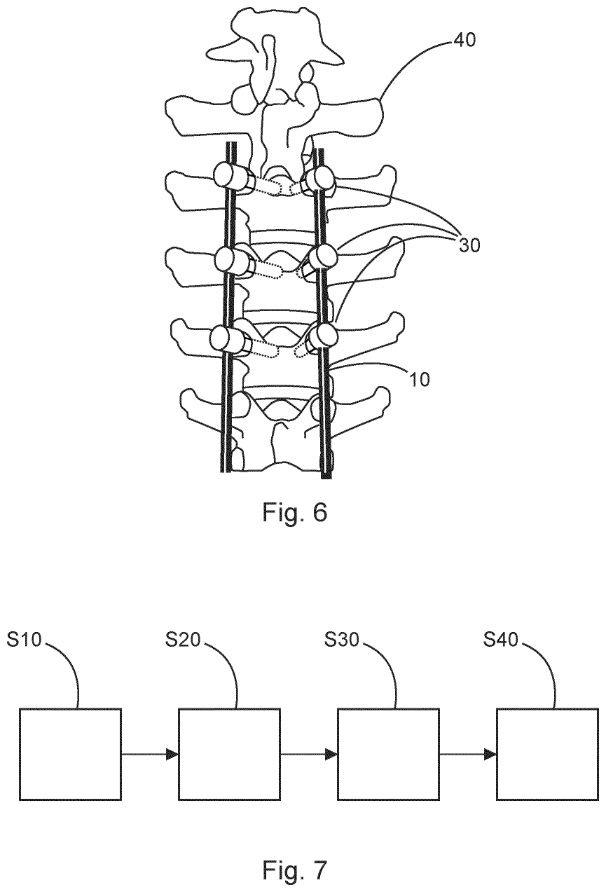

FIG. 6 shows a schematic view of a spine 40 of a patient 70 with spinal screws 30 that are connected by a spinal rod 10. The spinal screws 30 are inserted into the vertebral bodies, in particular pedicles or massa lateralis and thus are directly connected to the spine 40 of the patient. FIG. 6 illustrates that in general two parallel rows of spinal screws 30 are inserted in the spine 40 and each row of spinal screws 30 is connected with one spinal rod 10.

FIG. 7 shows a schematic view of the computer implemented method for augmented reality spinal rod planning and bending for navigated spine surgery. In a first step S10, a position Ps of a plurality of spinal screws 30 disposed on a spine 40 is acquired, wherein the plurality of spinal screws 30 are configured for receiving a spinal rod 10 interconnecting the plurality of spinal screws 30. In another step S20, a proposed spinal rod 20 is determined, being a virtual model of a spinal rod 10 with a desired shape using the acquired position Ps of the plurality of spinal screws 30. In another step S30, the spinal rod 10 is calibrated for tracking the spinal rod 10 by a medical navigation device 50. In another step S40, the proposed spinal rod 20 is displayed, by an augmented reality device 53, thereby overlaying the tracked spinal rod 10 with the proposed spinal rod 20.

1. A computer implemented method for augmented reality spinal rod planning and bending for navigated spine surgery, comprising:
acquiring a position of a plurality of spinal screws disposed on a spine, wherein the plurality of spinal screws are configured for receiving a spinal rod interconnecting the plurality of spinal screws;
determining a proposed spinal rod, being a virtual model of a spinal rod with a desired shape, using the acquired position of the plurality of spinal screws;
calibrating the spinal rod for tracking the spinal rod by a medical navigation device; and
displaying the proposed spinal rod, by an augmented reality device, thereby overlaying the tracked spinal rod with the proposed spinal rod.

2. The method of claim 1,
wherein the proposed spinal rod comprises a shape matching the position of the plurality of spinal screws on the spine.

3. The method of claim 1, comprising
determining the proposed spinal rod using the acquired position of the plurality of spinal screws and a planned shape of the spine.

4. The method of claim 1, wherein
calibrating the spinal rod comprises:
determining a spinal rod model, being a virtual representation of the spinal rod;
wherein the method further comprises the step of:
determining support data using the spinal rod model;
wherein the support data comprises information linked to the spine; and
overlaying, by the augmented reality device, the spinal rod with the support data.

5. The method of claim 4, wherein determining the spinal rod model, comprises:
recognizing the shape of the spinal rod in relation to a tracked reference array.

6. The method of claim 4, wherein determining the spinal rod model, comprises:
acquiring the shape of the spinal rod by a tracking device.

7. The method of claim 1, comprising the step:
dynamically adjusting the spinal rod model using the tracked spinal rod.

8. The method of claim 4,
wherein the support data comprises at least one bending indicator, determined by using the proposed spinal rod and the spinal rod model.

9. The method of claim 4, comprising the steps:
determining a spine model, being a virtual representation of the spine; and
adjusting the spine model using the spinal rod model;
wherein the support data comprises a spine indicator, determined by using the spine model, indicating the spine on the spinal rod.

10. The method of claim 4,
determining a spinal screw model, being a virtual representation of the plurality of spinal screws disposed on the spine;
wherein the support data comprises at least one screw indicator, determined by using the position of the plurality of spinal screws, indicating the plurality of spinal screws on the spinal rod.

11. The method of claim 4, comprising the step:
determining forces applied to the plurality of spinal screws by using the spine model and the spinal rod model;
wherein the support data comprises a force indicator, determined by using the determined forces, indicating forces applied to the plurality of spinal screws, if when the spinal rod is connected to the spinal screws.

12. The method of claim 4, comprising the step:
determining a force warning, if when the determined forces exceed a predetermined threshold;
wherein the support data comprises a force warning indicator, determined by using the determined force warning.

13. The method of claim 4, comprising the step:
determining at least one anatomical parameter of the spine by using the spine model and the spinal rod model;
wherein the support data comprises at least one anatomical parameter indicator, determined by using the at least one determined anatomical parameter.

14. Method of claim 4,
determining an average deviation between the spinal rod and the proposed spinal rod by using the spinal rod model and the proposed spinal rod;
wherein the support data comprises a deviation indicator, determined by using the determined average deviation.

15. The method of claim 1,
wherein calibrating the spinal rod comprises:
providing the spinal rod with a reference device, defining an origin of a spinal rod coordinate system;
determining a spinal-rod-to-cam-coordinate-transformation, which describes a transformation between the spinal rod coordinate system and a camera coordinate system.

16. The method of claim 1,
wherein acquiring the position of the plurality of spinal screws comprises recognizing the plurality of spinal screws by the augmented reality device.

17. The method of claim 1,
wherein acquiring the position of the plurality of spinal screws comprises extracting of the position of the plurality of spinal screws from a planning application.

18. The method of claim 1,
wherein acquiring the position of the plurality of spinal screws comprises detecting the position of the plurality of spinal screws in intraoperative paired image data.

19. The method of claim 1,
wherein acquiring the position of the plurality of spinal screws comprises calibrating each of the plurality of spinal screws by using a tracked pointer.

20. (canceled)

21. A non-transitory computer readable medium comprising instructions for augmented reality spinal rod planning and bending for navigated spine surgery which, when running on a computer or when loaded onto a computer, causes the computer to perform the steps of:
acquiring a position of a plurality of spinal screws disposed on a spine, wherein the plurality of spinal screws are configured for receiving a spinal rod interconnecting the plurality of spinal screws;
determining a proposed spinal rod, being a virtual model of a spinal rod with a desired shape, using the acquired position of the plurality of spinal screws;
calibrating the spinal rod for tracking the spinal rod by a medical navigation device; and
displaying the proposed spinal rod, by an augmented reality device, thereby overlaying the tracked spinal rod with the proposed spinal rod.
